(19) United States
(12) Patent Application Publication
(10) Pub. No.: US 2023/0078164 A1
(43) Pub. Date: Mar. 16, 2023

(54) PHRENIC NERVE STIMULATION WITH MECHANICAL VENTILATION

(71) Applicant: Covidien LP, Mansfield, MA (US)

(72) Inventors: Abhijit BHATTACHARYA, Hyderabad (IN); Lilian KORNET, Eijsden (NL); Avram SCHEINER, Vadnais Heights, MN (US)

(73) Assignee: Covidien LP, Mansfield, MA (US)

(21) Appl. No.: 17/870,564

(22) Filed: Jul. 21, 2022

Related U.S. Application Data
(60) Provisional application No. 63/241,747, filed on Sep. 8, 2021.

(51) Int. Cl. A61N 1/05; A61N 1/36; A61M 16/00

(52) U.S. Cl. CPC A61N 1/0519; A61N 1/3611; A61N 1/36139; A61N 1/36146; A61M 16/024; A61M 2025/1043

(57) ABSTRACT

Aspects of this disclosure describe methods and systems for nerve stimulation using a balloon catheter. The balloon catheter includes a catheter, an inflatable balloon with a surface, and a set of electrodes positioned along the surface of the inflatable balloon. The balloon catheter may be positioned in a vessel of a patient, such as the esophagus. The patient may be concurrently undergoing mechanical ventilation. The balloon catheter is secured in the vessel by inflating the inflatable balloon. When the inflatable balloon is inflated, the surface of the inflatable balloon and the set of electrodes is positioned at an internal wall of the vessel. Stimulation is provided to a nerve near the vessel, via the set of electrodes, based on stimulation parameters. Values for the stimulation parameters may be adjusted based on breathing parameters of the patient. The stimulation parameters may also be adjusted to wean a patient off mechanical ventilation.

### CROSS-REFERENCE TO RELATED APPLICATIONS

This application claims the benefit of U.S. Provisional Application No. 63/241,747 filed Sep. 8, 2021, titled “Phrenic Nerve Stimulation with Mechanical Ventilation,” which is incorporated herein by reference in its entirety.

### INTRODUCTION

Medical ventilator systems have long been used to provide ventilatory and supplemental oxygen support to patients. These ventilators typically include a connection for pressurized gas (air, oxygen) that is delivered to the patient through a conduit or tubing. As each patient may require a different ventilation strategy, modern ventilators can be customized for the particular needs of an individual patient. For example, several different ventilator modes or settings have been created to provide better ventilation for patients in different scenarios, such as mandatory ventilation modes, spontaneous ventilation modes, and assist-control ventilation modes. Ventilators monitor a variety of patient parameters and are well equipped to provide reports and other information regarding a patient's condition.

Long term external ventilation is typically provided to patients using positive pressure ventilation. Positive pressure ventilation is a form of artificial respiration in which a mechanical ventilator is used to deliver a controlled volume of gasses to the lungs of a patient. In contrast, in one form of negative-pressure ventilation, the diaphragm of a patient is caused to contract to cause the chest of the patient to expand during inspiration (thereby drawing air into the lungs), and the diaphragm is caused to relax to cause the chest to contract during exhalation (thereby forcing air out of the lungs). While lifesaving and valuable, positive pressure ventilation is non-physiological; that is, forcing air into the lungs is not the manner in which humans naturally breathe. Accordingly, the greater the positive pressure and/or the number of positive-pressure cycles, the more likely the patient will experience detrimental effects, such as an illness becoming more severe, ventilator-induced lung injury, acute respiratory distress syndrome (ARDS), ventilator-associated pneumonia (VAP), diaphragm dystrophy, and delay of ventilator weaning. These detrimental effects may increase an amount of time a patient is subjected to mechanical ventilation, leading to longer hospital stays and increased medical costs.

It is with respect to this general technical environment that aspects of the present technology disclosed herein have been contemplated. Furthermore, although a general environment is discussed, it should be understood that the examples described herein should not be limited to the general environment identified herein.

### Phrenic Nerve Stimulation with Mechanical Ventilation

Among other things, aspects of the present disclosure include systems and methods for phrenic nerve stimulation with mechanical ventilation. This disclosure describes systems and methods for phrenic nerve stimulation (PNS) during mechanical ventilation of a patient or as an alternative to mechanical ventilation. Additionally, this disclosure describes pacing of the phrenic nerve from the esophagus.

In an aspect, a balloon catheter is disclosed. The balloon catheter includes a catheter and an inflatable balloon positioned along a length of the catheter, the inflatable balloon having a surface. The balloon catheter further includes a set of electrodes on the surface of the inflatable balloon, the set of electrodes including a first electrode and a second electrode, the set of electrodes capable of stimulating a nerve. Additionally, the balloon catheter includes at least one wire running along the length of the catheter capable of providing power to the set of electrodes.

In an example, the first electrode and the second electrode are spaced apart on the surface of the inflatable balloon. In another example, the first electrode and the second electrode are spaced apart radially on the surface of the inflatable balloon. In a further example, the balloon catheter further includes a balloon port to inflate the inflatable balloon; an injection port to introduce a substance into the catheter; and a power port electrically coupled to the set of electrodes via the at least one wire. In yet another example, the set of electrodes further includes a third electrode and a four electrode. In still a further example, the balloon catheter further includes four of the set of electrodes. In another example, the four sets of electrodes are distributed along the surface of the inflatable balloon. In a further example, the first electrode and the second electrode are independently controllable.

In another aspect, a method for stimulating a nerve is disclosed. The method includes providing first nerve stimulation to a patient under mechanical ventilation, wherein the first nerve stimulation is based on first values for a set of stimulation parameters. After providing the first nerve stimulation to the patient, the method includes receiving a breathing parameter of the patient. Based on the breathing parameter, the method includes determining second values for the set of stimulation parameters. Additionally, based on the second values for the set of stimulation parameters, the method includes providing second nerve stimulation to the patient.

In an example, the set of stimulation parameters includes a frequency of stimulation, or an amplitude of stimulation, or both. In another example, the breathing parameter of the patient includes at least one of a tidal volume of the patient, an end-tidal CO₂, or a patient effort. In a further example, the method further includes determining that the breathing parameter exceeds a threshold; and based on determining that the breathing parameter exceeds the threshold, changing at least one of the frequency of stimulation or the amplitude of stimulation. In yet another example, the method further includes implementing a weaning maneuver, wherein the weaning maneuver includes: pausing providing nerve stimulation; and during the pause, receiving an indication of a patient effort, wherein the second values for the set of stimulation parameters is based on the patient effort. In still a further example, the breathing parameter of the patient is based on the stimulation parameters. In another example, the first nerve stimulation and the second nerve stimulation stimulate a phrenic nerve and are provided from inside an esophagus of the patient. In another example, the method further includes: based on the breathing parameter, stopping delivery of ventilation while maintaining phrenic nerve stimulation. In a further example, a frequency of the phrenic nerve stimulation is based on the detected patient effort.

In a further aspect, a ventilator for providing mechanical ventilation and nerve stimulation to a patient is disclosed. The ventilator includes a display, an inhalation port for providing breathing gas to the patient, a nerve stimulation port, a processor, and memory storing instructions that, when executed by the processor, cause the ventilator to perform a set of operations. The set of operations includes detecting a perceived patient effort and delivering the breathing gas to the patient via the inhalation port, based on the perceived patient effort. Additionally, the set of operations includes providing power to a set of electrodes coupled to an esophageal tube via the nerve stimulation port, wherein the power has a frequency and an amplitude. The set of operations also includes measuring a breathing parameter of the patient, wherein the breathing parameter includes a tidal volume of the patient, an end-tidal CO₂ of the patient, or both. Based on the breathing parameter of the patient, the set of operations includes adjusting at least one of the frequency, the amplitude, or both.

In an example, the set of operations further includes pausing providing power to the set of electrodes; and while pausing providing power, detecting an unassisted patient effort, wherein adjusting the at least one of the frequency, the amplitude, or both is further based on the unassisted patient effort. In another example, adjusting at least one of the frequency, the amplitude, or both is further based on the perceived patient effort. In a further example, the breathing gas is delivered according to a patient support mode, based on the patient effort. In another example, the esophageal tube includes an inflatable balloon and wherein the set of electrodes is positioned along the inflatable balloon.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 shows an anatomy of a human patient, including phrenic nerves.

FIGS. 2A-2D show different perspectives of an example balloon catheter for phrenic nerve stimulation.

FIGS. 3A-3D show different configurations of electrodes coupled to an inflatable balloon of a balloon catheter.

FIG. 4A shows an example placement of a balloon catheter in a patient for phrenic nerve stimulation.

FIG. 4B shows another example placement of a balloon catheter in a patient for phrenic nerve stimulation.

FIG. 4C shows a cross-sectional view of a balloon catheter and a phrenic nerve.

FIG. 5A shows an example method for phrenic nerve stimulation.

FIG. 5B shows an example flowchart for phrenic nerve stimulation.

FIG. 5C shows an example capnography.

FIG. 6 shows another example method for phrenic nerve stimulation.

FIG. 7 shows another example method for phrenic nerve stimulation.

FIG. 8 is a diagram illustrating an example of a ventilator connected to a human patient and a balloon catheter for phrenic nerve stimulation.

FIG. 9 is a block-diagram illustrating an example of a ventilatory system.

FIG. 10 is an example system for phrenic nerve stimulation.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, medical ventilators are used to provide breathing gases to patients who are otherwise unable to breathe sufficiently. In modern medical facilities, pressurized air and oxygen sources are often available from wall outlets, tanks, or other sources of pressurized gases. Accordingly, ventilators may provide pressure regulating valves (or regulators) connected to centralized sources of pressurized air and pressurized oxygen. The regulating valves function to regulate flow so that respiratory gases having a desired concentration are supplied to the patient at desired pressures and flow rates.

Approximately one third of patients in the intensive care unit depend on mechanical ventilation. With millions of people each year admitted to the intensive care unit, many people per year rely on mechanical ventilation. Additionally, respiratory illnesses such as SARS-CoV-2 (otherwise referred to as COVID-19) increase the number of individuals depending on mechanical ventilation. The amount of time a patient is assisted via mechanical ventilation may vary, as may be based on a lung condition of the patient. For example, approximately a third of patients with COPD or ARDS may seek assistance from a mechanical ventilator for longer than 4 days, with some patients requiring mechanical ventilation longer than seven days. Additionally, patients with sleep apnea may require mechanical ventilation while sleeping, over an extended period of time.

Weaning patients off from mechanical ventilation may be difficult. A patient's diaphragm muscles may begin to atrophy after as little as two days of ventilation. After a patient's diaphragm muscles begin to atrophy, a patient may require slow weaning to encourage the patient to breathe on their own (e.g., via the patient's diaphragm contracting unassisted by a mechanical ventilator). Increased time on a ventilator is associated with increased risk of infection, hyperventilation, hypoventilation, decreased venous return, and subsequent rehospitalization. Thus, minimizing a patient's time on a ventilator may be desirable for diaphragm health and/or to reduce the risk of infection and/or rehospitalization.

One solution is to exercise the patient's diaphragm muscles during ventilation to prevent or reduce the likelihood of diaphragm muscle atrophy and reduce the time that a patient is dependent on mechanical ventilation. To exercise a patient's diaphragm, the phrenic nerve can be stimulated with an electrical current. The electrical current to stimulate the phrenic nerve may be provided by electrodes producing a nearby electrical current. The electrodes may be positioned inside of the body of the patient close enough to the phrenic nerve to stimulate the phrenic nerve. For example, electrodes may be positioned on a device that is insertable into the body of the patient. In one example, electrodes may be positioned on a lead insertable into a blood vessel (e.g., vein, artery, arteriole, capillary, venule, etc.) near a phrenic nerve. Intravenous placement of a lead, however, may cause complications, including infection. Additionally, some respiratory clinicians may not be skilled with placement of a lead in a blood vessel, resulting in placement issues and/or complications. Examples of stimulating one or both phrenic nerves with an intravenous stimulation lead are provided in U.S. patent application Ser. No. 17/039,115, titled “Intravenous Phrenic Nerve Stimulation Lead” and filed on Sep. 30, 2020, which is incorporated herein by reference in its entirety.

In another example, electrodes may be integrated into a structure of a catheter. The catheter is insertable into an orifice in the body of the patient. In an example, electrodes are positioned along an inflatable balloon of a balloon catheter. The balloon catheter may be an esophageal balloon catheter that is capable of providing nutrients to a patient through the esophagus of the patient. The balloon catheter may be secured in the esophagus with an inflatable balloon. Unlike an intravenous lead, for which some respiratory clinicians may not be skilled with intravenous placement in a body of a patient, placement of a catheter in an esophagus of a patient is commonly performed by respiratory clinicians. For example, patients undergoing invasive mechanical ventilation and/or sedation commonly require or desire an esophageal catheter to provide nutrients to sustain the patient.

Pacing therapy of a phrenic nerve (sometimes referred to as phrenic nerve pacing or diaphragm pacing) may prevent or reverse diaphragm muscle-disuse atrophy, maintain diaphragmatic endurance, and facilitate weaning of patients from mechanical ventilation. Phrenic nerve pacing may be caused by stimulating the phrenic nerve to thereby cause the phrenic nerve to send an electrical impulse to the diaphragm. The paced diaphragm is expected to restore negative-pressure ventilation, thereby potentially providing a more physiological respiratory pattern and reducing the levels of positive pressure ventilation and its harmful effects on the lungs. Additionally, keeping the diaphragm active retains a patient's ability to cough, which reduces secretion and infection. Phrenic nerve pacing may be used for patients experiencing chronic spinal injury or other injury that may benefit from pacing a few hours per day (or otherwise infrequently or non-continuously).

Aspects of this disclosure describe a balloon catheter. The balloon catheter includes a catheter and an inflatable balloon. Electrodes are coupled to the inflatable balloon. The electrodes may be capable of stimulating a phrenic nerve from within a vessel in a body of a patient. In an example, the balloon catheter is positioned in an esophagus of a patient near one or both phrenic nerves. Aspects of the electrodes may be independently controllable.

In another aspect, a method for stimulating a phrenic nerve is disclosed. A patient receiving invasive mechanical ventilation (IMV) may be monitored to determine muscle activity. After muscle activity is detected, nerve stimulation is provided to support the patient in addition to IMV (e.g., ventilating the patient using both negative pressure ventilation and positive pressure ventilation). The ventilator may provide ventilatory support to the patient as required or desired in addition to the support caused by nerve stimulation. If no ventilatory support is required or desired in addition to the nerve stimulation, then nerve stimulation is provided without IMV. Breathing parameters of the patient are monitored, such as end expiratory CO₂ (also referred to herein as end-tidal CO₂) or tidal volume. Nerve stimulation parameters are adjusted based on the breathing parameters. Nerve stimulation parameters may include amplitude (e.g., amount of voltage provided to one or more electrodes) and frequency (e.g., number of stimulation pulses per time). The patient may be weaned from nerve stimulation and/or IMV over time.

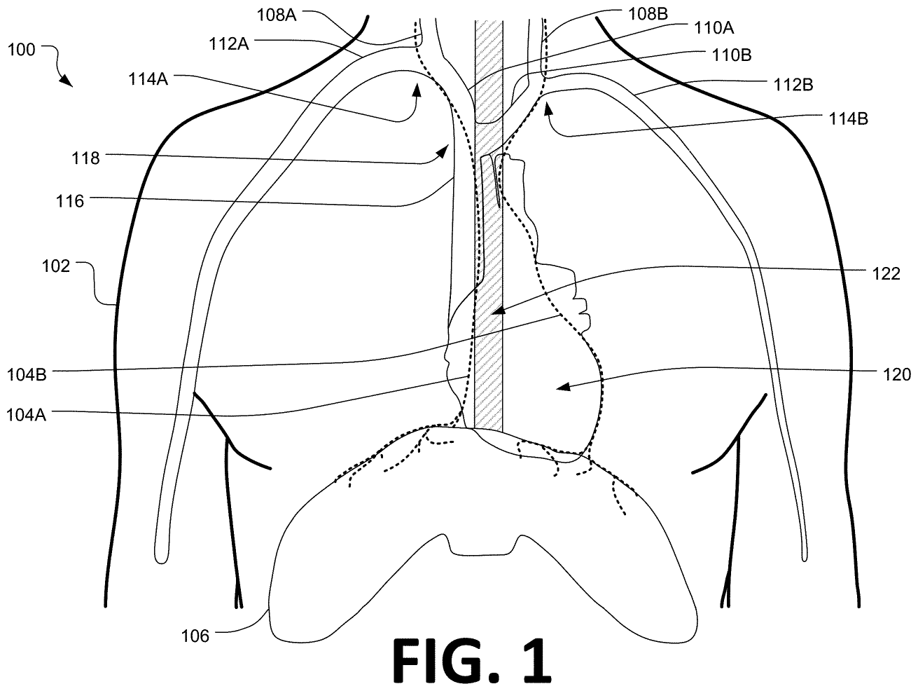

FIG. 1 shows an anatomy of a human patient 100, including phrenic nerves (i.e., right phrenic nerve 104A and left phrenic nerve 104B). The body 102 of the patient 100 includes a right phrenic nerve 104A, a left phrenic nerve 104B, a diaphragm 106, a right internal jugular vein 108A, a left internal jugular vein 108B, a right brachiocephalic vein 110A, a left brachiocephalic vein 110B, a right subclavian vein 112A, a left subclavian vein 112B, a right jugular-brachiocephalic junction 114A, a left jugular-brachiocephalic junction 114B, a superior vena cava (SVC) 116, an SVC junction 118, a heart 120, and an esophagus 122. It should be appreciated that, although not shown, the body 102 of the patient 100 contains other anatomical structures, including a stomach fed by the esophagus 122.

The right phrenic nerve 104A and the left phrenic nerve 104B originate from the spinal cord in the neck region (C3-C5 cervical vertebral region). The right phrenic nerve 104A extends through the body 102 from the right side of the neck region between the right lung and the heart 120 to the right side of the diaphragm 106. The left phrenic nerve 104B extends through the body 102 from the left side of the neck region between the left lung and the heart 120 to the left side of the diaphragm 106. The right phrenic nerve 104A and the left phrenic nerve 104B may each, independently, send motor information to the diaphragm 106 using an electrical signal. The phrenic nerve is “stimulated” when the phrenic nerve sends an electrical signal to the diaphragm 106. To “effectively stimulate” the phrenic nerve, one or more muscles of the diaphragm 106 move (such as stiffening or contracting) due to the electrical signal sent from the phrenic nerve. The electrical signal to stimulate the phrenic nerve may originate naturally from the brain or may be provided artificially (e.g., by a nearby electrical current, and resulting magnetic field, from electrodes on a catheter). For example, the right phrenic nerve 104A and/or the left phrenic nerve 104B may be artificially stimulated by a magnetic field resulting from an electrical current between one or more electrodes on a catheter (such as an esophageal balloon catheter). As an artificial magnetic field is created near the phrenic nerve, transmission fibers of the phrenic nerve are excited to create movement of muscles in the diaphragm. For the magnetic field to effectively stimulate the phrenic nerve, the one or more electrodes carrying the electrical current should be placed near, adjacent, or proximate the right phrenic nerve 104A and/or the left phrenic nerve 104B.

The motor information sent to the diaphragm 106 at the right phrenic nerve 104A and/or the left phrenic nerve 104B causes movement of one or more muscles of the diaphragm 106. Muscle movement of the diaphragm may cause expansion of the lungs of the patient. For example, stimulation of the right phrenic nerve 104A causes one or more muscles on a right portion of the diaphragm 106 to move, thereby causing expansion of one or both lungs of the patient 100. In another example, stimulation of the left phrenic nerve 104B causes one or more muscles on a left portion of the diaphragm 106 to move, thereby causing expansion of one or both lungs of the patient 100. Stimulation of only one of the phrenic nerves (i.e., right phrenic nerve 104A or left phrenic nerve 104B) may cause muscle movement on both sides of the diaphragm, due to the collective nature of the diaphragm muscles (e.g., stimulating muscles on one side will also move some of the muscles on the other side). For some patients, stimulation of only one of the phrenic nerves is sufficient, while other patients may require or desire stimulation of both phrenic nerves. Examples of stimulating one or both phrenic nerves to obtain tidal volumes are provided in U.S. patent application Ser. No. 16/888,960, titled “Achieving Smooth Breathing by Modified Bilateral Phrenic Nerve Pacing,” and filed on Jun. 1, 2020, which is incorporated herein by reference in its entirety.

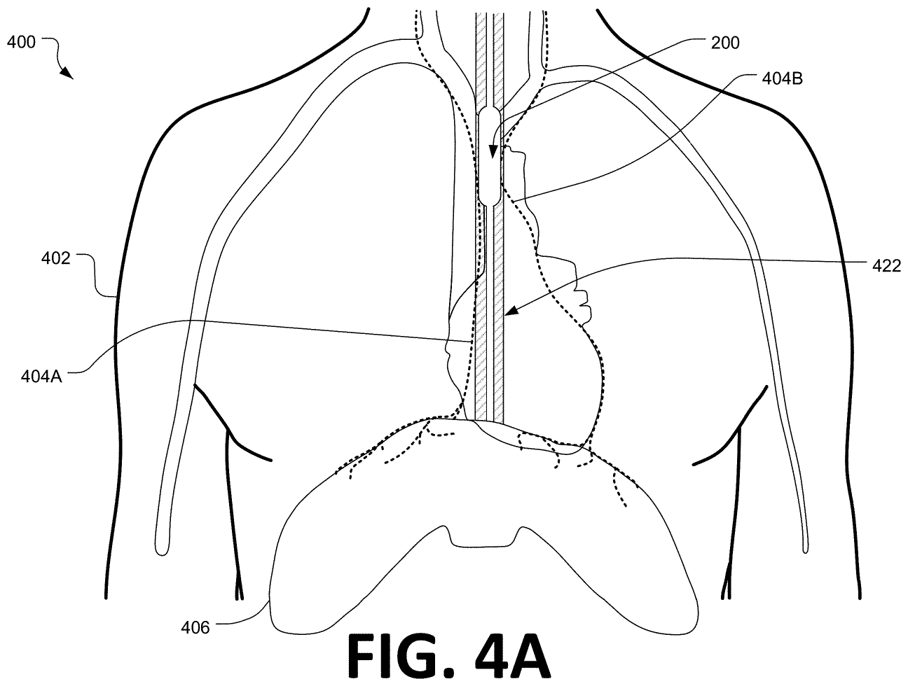

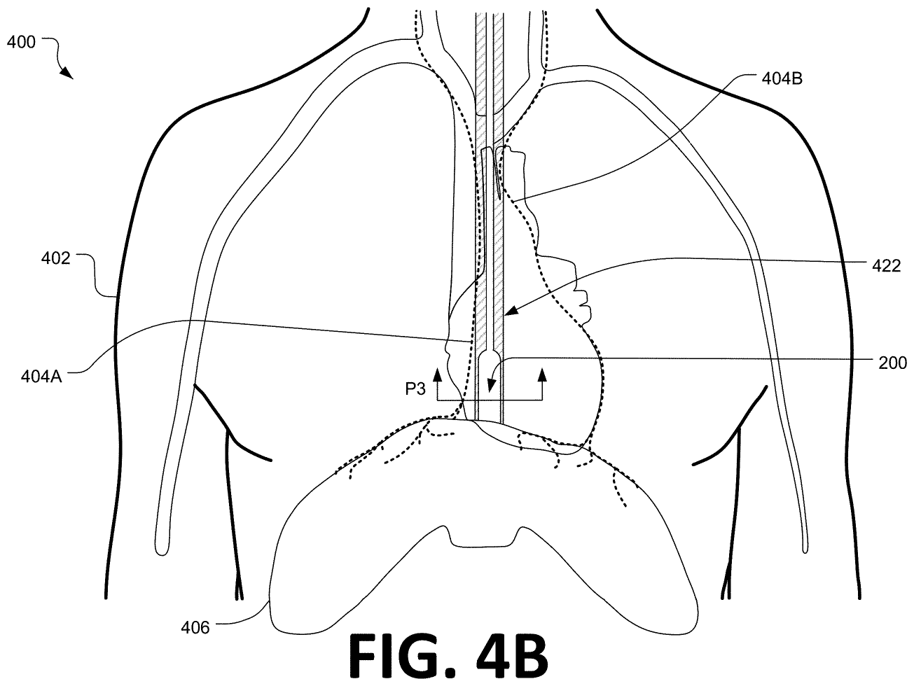

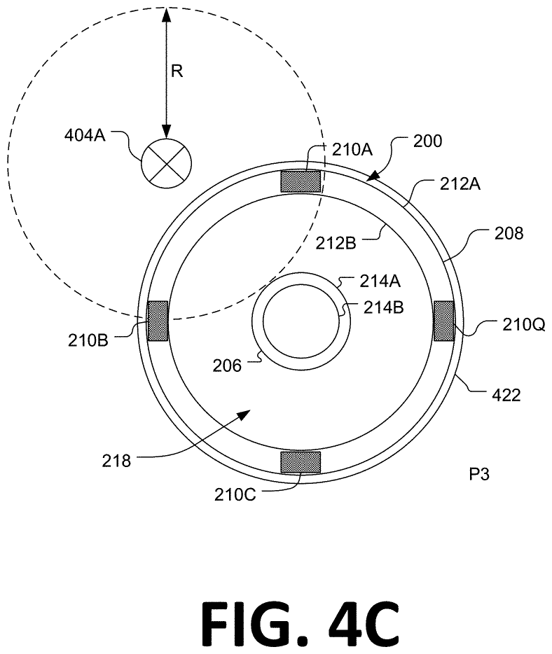

In an example, a phrenic nerve (e.g., the right phrenic nerve 104A and/or left phrenic nerve 104B) may be stimulated by a magnetic field caused by a current between two or more electrodes. The electrodes may be positioned inside an esophagus 122 of the patient 100 that runs proximate to a phrenic nerve, such that the phrenic nerve is inside the resulting magnetic field. Placement of the electrodes on a catheter in the esophagus 122 is further described with respect to FIGS. 4A-4C. The one or more electrodes may be placed on or along an inflatable balloon of a catheter, as described herein.

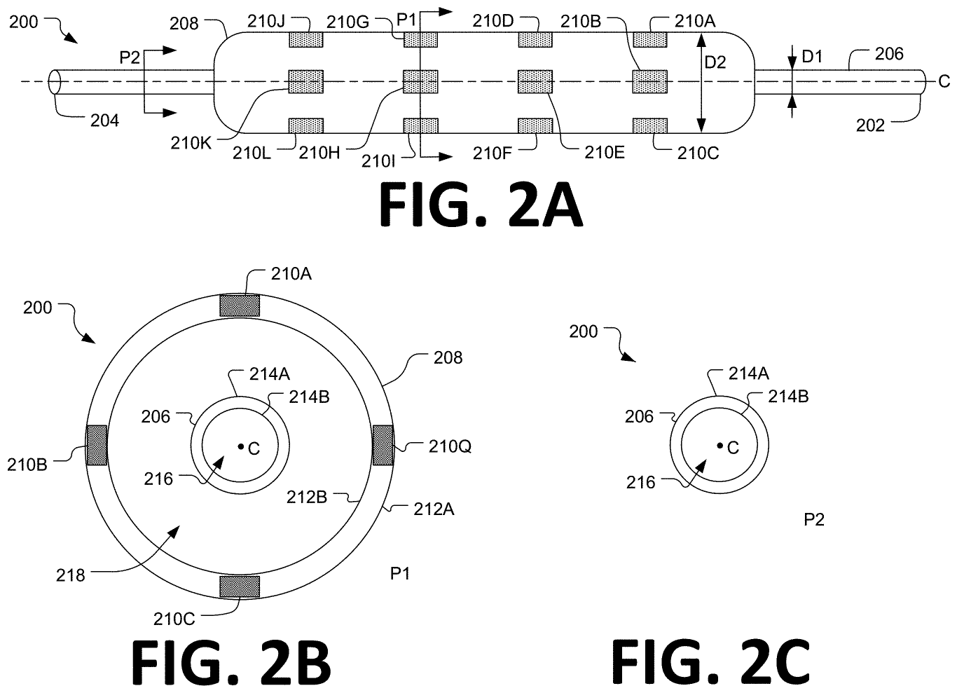

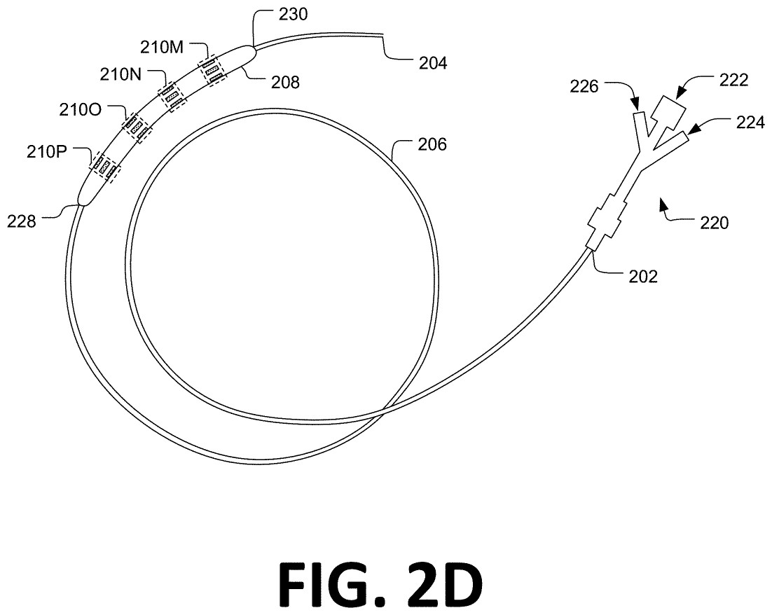

FIGS. 2A-2D show an example of a balloon catheter 200. Specifically, FIG. 2A shows a side perspective view of the inflatable balloon 208 of the balloon catheter 200. FIG. 2B shows a cross-sectional view of the balloon catheter 200 at viewing plane P1. FIG. 2C shows a cross-sectional view of the balloon catheter 200 at viewing plane P2. FIG. 2D shows the balloon catheter 200 including the inflatable balloon 208 and a connector 220. As shown in FIGS. 2A-2D, the balloon catheter 200 includes a catheter 206, an inflatable balloon 208, and a connector 220.

The catheter 206 includes a first end 202 and a second end 204. The catheter 206 also includes an exterior surface 214A and an interior surface 214B. The exterior surface 214A of the catheter 206 may be a cylinder with a catheter diameter D1. Although a cylindrical shape of the catheter 206 is shown in FIGS. 2A-2D, other shapes are appreciated, such as a rectangular prism, n-sided prism, ovular cylinder, etc. The interior surface 214B of the catheter 206 defines a catheter annulus 216 extending from the first end 202 to the second end 204. The catheter annulus 216 is capable of providing fluid or fluid-like substances from the first end 202 to the second end 204 or vice versa. The catheter annulus 216 may be fluidly coupled with an injection port 222 of a connector 220. For example, a substance may be introduced into the catheter annulus 216 via an injection port 222 of the balloon catheter 200. The catheter annulus 216 may have a similar shape as that defined by the exterior surface 214A of the catheter 206. For example, the catheter annulus 216 may be a cylinder with a diameter smaller than the catheter diameter D1. The catheter 206 may be made of a flexible, rigid, or semi-rigid material. In an example, the catheter 206 is made of plastic, which may be medical grade.

The inflatable balloon 208 includes a first end 228 and a second end 230. Both the first end 228 and the second end 230 of the inflatable balloon 208 are positioned along the length of the catheter 206 between the first end 202 and the second end 204. The inflatable balloon 208 may share a center C with the catheter 206. The inflatable balloon 208 also includes an exterior surface 212A, an interior surface 212B, an inflatable annulus 218, and one or more electrodes 210A-210L. The exterior surface 212A of the inflatable balloon 208 may be a cylinder with rounded edges at the first end 228 and the second end 230. The inflatable balloon 208 may or may not have rounded edges at the first end 228 and/or the second end 230. In the example shown, the cylindrical portion of the inflatable balloon 208 (e.g., such as the portion of the inflatable balloon 208 shown at the viewing plane P1 in FIG. 2B) may have a balloon diameter D2. The balloon diameter D2 is variable based on how much the inflatable balloon 208 is inflated. For example, when the inflatable annulus is filled or partially filled with gas, the balloon diameter D2 is larger than the catheter diameter D1. Although a cylindrical shape of a portion of the inflatable balloon 208 is shown in FIGS. 2A-2D, other shapes are appreciated, such as a rectangular prism, n-sided prism, ovular cylinder, etc.

The inflatable annulus 218 is defined by the interior surface 214B of the inflatable balloon 208 and the exterior surface 214A of the catheter 206. For example, the inflatable balloon 208 may be positioned along the catheter 206 and enclose a portion of the catheter 206 from the first end 228 of the inflatable balloon 208 to the second end 230 of the inflatable balloon 208. The inflatable annulus 218 is configured to be inflated to change the balloon diameter D2. The inflatable annulus 218 may be fluidly coupled with a balloon port 224 of a connector 220. For example, a gas may be introduced into the inflatable annulus 218 via a balloon port 224 of the balloon catheter 200. The balloon port 224 may include a valve to releasably maintain gas in the inflatable annulus 218. The inflatable balloon 208 may be made of a flexible, rigid, or semi-rigid material. In an example, the inflatable balloon 208 is made of plastic, which may be medical grade. In another example, the inflatable balloon 208 and the catheter 206 are the same material. The thickness of the material of the inflatable balloon 208 (e.g., the distance between the interior surface 212A and exterior surface 212B of the inflatable balloon 208) may be thinner than the thickness of the material of the catheter 206 (e.g., the distance between the interior surface 214A and exterior surface 214B of the catheter 206). A thinner material may be more flexible than a thicker material.

The inflatable balloon 208 may also include one or more electrodes 210A-210L. the electrodes 210A-210L may be coupled to the inflatable balloon 208. For example, the electrodes 210A-210L may be coupled to the exterior surface 212A of the inflatable balloon 208, the interior surface 212B of the inflatable balloon 208, or positioned between the exterior surface 212A and the interior surface 212B of the inflatable balloon 208. The coupling of the electrodes to the inflatable balloon 208 may cause the electrodes to be positioned along the balloon diameter D2 of the inflatable balloon D2 at any inflation capacity (e.g., inflated, partially inflated, deflated) of the inflatable annulus 218. One or more attributes of each electrode 210A-210L may be unique and/or independently controllable. For example, attributes of each electrode may include polarity, pulse length, pulse frequency, amplitude, etc. To cause an electrical current, two or more electrodes may be assigned opposite polarities. The electrodes may independently communicatively couple and/or electrically couple to one or more controllers. For example, an electrical coupling to each electrode may be provided at a power port 226 of the connector 220 of the balloon catheter 200. The electrical coupling to each electrode may be via one or more wires. The controller may be electrically coupled to a power source. When voltage is applied, a current flows between at least two electrodes. A power source may be an external battery that may be replaceable and/or rechargeable, or the power source may be shared with an external device (e.g., a power source of a ventilator). For example, the electrodes on the balloon catheter 200 may be electrically coupled to an external device that provides power to the electrodes. In another example, a ventilator may provide power to the balloon catheter 200 while contemporaneously ventilating a patient. It should be appreciated that the balloon catheter may include one or more inflatable balloons 208, which may be independently inflatable and which may or may not each include electrodes.

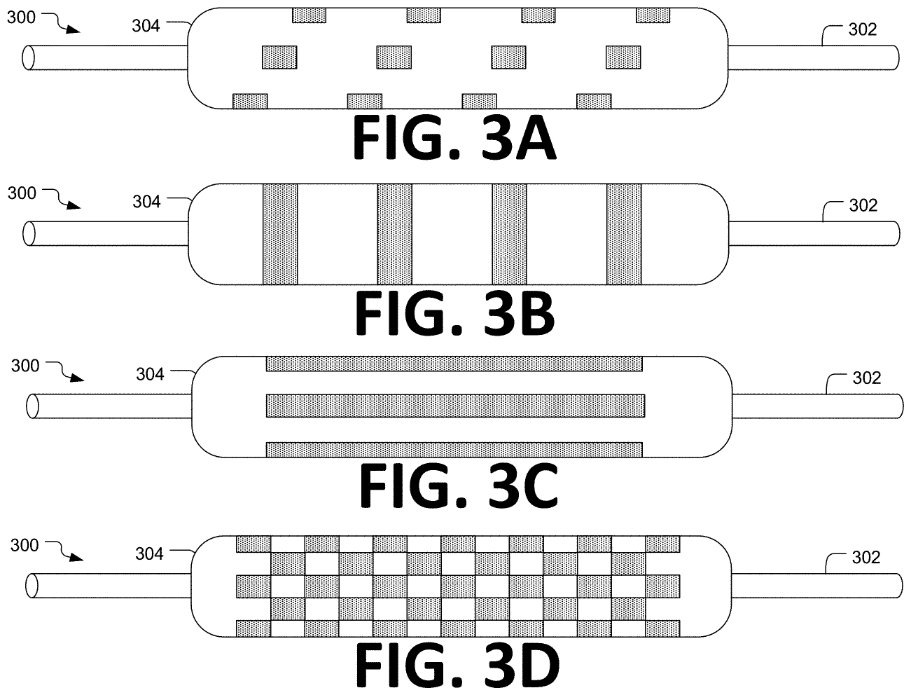

In the example shown in FIG. 2D, one or more electrodes are spaced apart along the exterior surface 212A of the inflatable balloon 208. The electrodes may be spaced apart radially on the exterior surface 212A of the inflatable balloon 208. In an example, four electrodes (e.g., electrodes 210A, 210B, 210C, 210Q) may be radially spaced near the exterior surface 212A of the inflatable balloon 208. The electrodes 210A-210L may be grouped into a set of one or more electrodes (e.g., electrode sets 210M-210P). As further shown in FIG. 2D, multiple sets of electrodes may be spaced along the length of the inflatable balloon 208 (e.g., between the first end 228 and the second end 230). In the example shown in FIG. 2D, the inflatable balloon 208 may include four sets of four electrodes. As shown, the four sets of electrodes 210M-210P are distributed along the exterior surface 212A of the inflatable balloon 208. In an instance, each set of electrodes may be aligned and oriented radially about the inflatable balloon 208 at a 90-degree angle from each other about the center C and each set of electrodes 210M-210P are evenly spaced lengthwise (e.g., between first end 228 and second end 230). Although a particular configuration of electrodes is shown in FIGS. 2A-2D, the electrodes may be configured and/or spaced in any way about the exterior surface 212A of the inflatable balloon 208. Other configurations are further described herein at least with respect to FIGS. 3A-3D.

As further described herein, the connector 220 includes an injection port 222, a balloon port 224, and a power port 226. The injection port 222 is fluidly coupled to the catheter annulus 216 of the catheter 206. The balloon port 224 is fluidly coupled to the inflatable annulus 218 of the inflatable balloon 208. The power port 226 of the connector 220 is electrically coupled to each electrode 210A-210L to allow independent control of each electrode 210A-210L via a controller and/or power source couped to the power port 226. The injection port 222 and/or the balloon port 224 may include a check valve to reduce or prevent back flow. For example, the injection port 222 may include a check valve to prevent back flow of a substance from the second end 204 of the catheter 206 out of the injection port 222. In another example, the balloon port 224 may include a check valve to maintain inflation of an inflatable annulus 218 of an inflatable balloon 208. For instance, the inflatable annulus 218 may be inflated to secure an exterior surface 212A of the inflatable balloon 208 to an interior wall of a vessel in a patient at or after the inflatable annulus 218 is inflated. A check valve of the injection port 222 and/or balloon port 224 may be releasable (e.g., to allow backflow). For example, the check valve may be released to deflate the inflatable annulus 218 prior to moving the balloon catheter 200 inside the patient.

The balloon catheter 200 is positionable inside the esophagus of a patient near a phrenic nerve. Placement of the balloon catheter 200 inside the body of the patient near a phrenic nerve may allow stimulation of the nearby phrenic nerve using one or more of the electrodes 210A-210L on the balloon catheter 200.

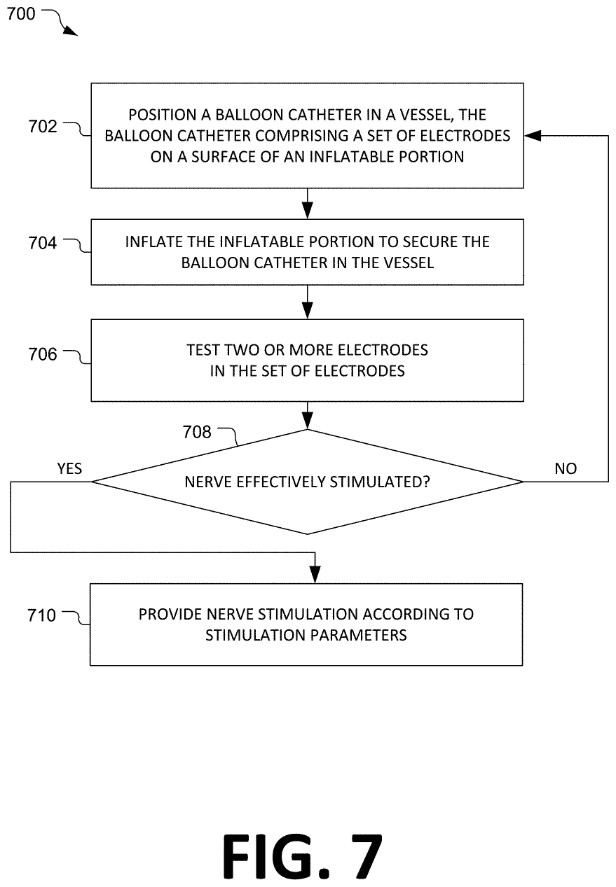

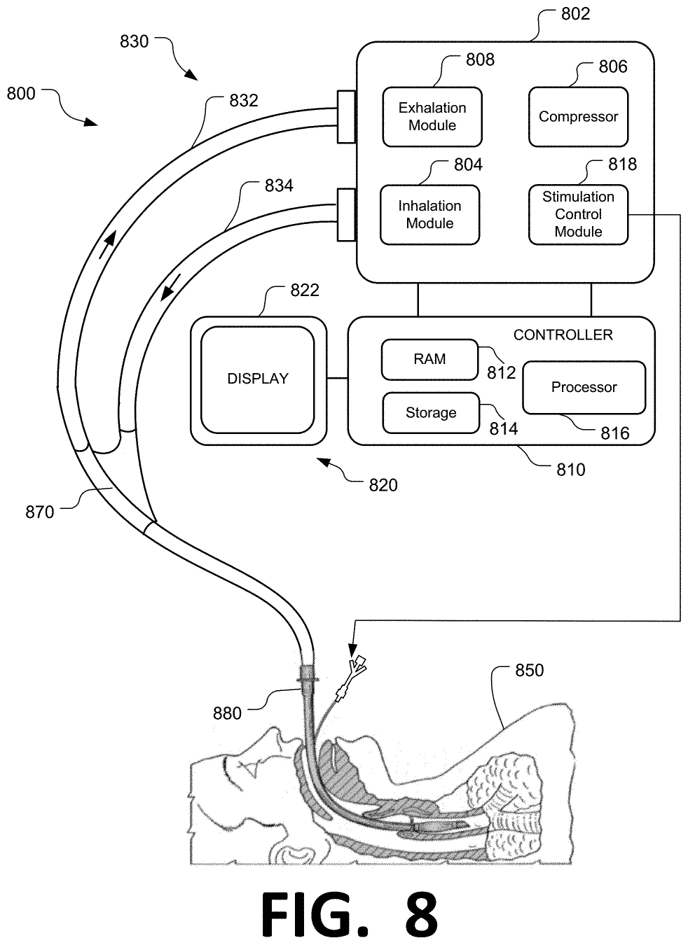

The balloon catheter 200 in a deflated configuration may be positioned inside the body prior to transitioning the inflated configuration, by inflating the inflatable balloon 208. For proper positioning, the balloon catheter 200 may be positioned and re-positioned inside the body while the electrodes 210A-210J provide an electrical current and resulting magnetic field, until the diaphragm muscles move due to stimulation of the phrenic nerve. For example, to determine a desired position of the balloon catheter 200 in a body of a patient, the balloon catheter is moved in the body, the inflatable balloon 208 of the balloon catheter 200 is inflated, and the electrodes of the balloon catheter are tested to determine if a phrenic nerve is effectively stimulated. This test may involve experimentation with one or more attributes of one or more electrodes. If the phrenic nerve is effectively stimulated, then the position of the balloon catheter 200 may be maintained. If the phrenic nerve is not effectively stimulated, then the process may be repeated (e.g., deflate the inflatable balloon 208, move the balloon catheter 200, inflate the inflatable balloon, and test the electrodes of the balloon catheter 200). Further discussion of determining positioning of the balloon catheter and attributes of the electrodes is further described at least with respect to FIG. 7.

When the balloon catheter 200 is in the inflated configuration, a surface of the inflatable balloon 208 may be inflated such as to apply pressure to an internal wall of a vessel (e.g., an esophagus). An inflatable annulus 218 of the balloon catheter 200 that is inflated may target a phrenic nerve better than an inflatable annulus 218 that is deflated. A phrenic nerve may be effectively stimulated when in the presence of a magnetic field. For a given current between at least two electrodes (e.g., electrodes 210A-210L), the phrenic nerve will be effectively stimulated within a stimulation radius of the electrical current (e.g., stimulation radius R shown in FIG. 4C). By distancing the electrodes 210A-210L from the center C of the balloon catheter 200, the current travelling between two or more electrodes 210A-210L is more likely to be within the stimulation radius of the phrenic nerve.

Additionally, inflation of the inflatable balloon 208 may cause contact of the exterior surface 212A of the inflatable balloon 208 with an interior wall of a vessel to secure or fixate the balloon catheter 200 in the body of the patient. For example, when the inflatable balloon 208 is inflated (e.g., the inflatable balloon 208 has a larger balloon diameter D2), the exterior surface 212A of the inflatable balloon 208 expands to contact the interior wall of a vessel at a pressure equal to the gas pressure inside the inflatable annulus 218. Contacting of the exterior surface 212A of the inflatable balloon 208 with an interior wall of a vessel provide stability and security of the balloon catheter 200 within the vessel and within the body of the patient.

FIGS. 3A-3D show different configurations of electrodes positioned on an inflatable balloon 304 positioned along a length of a catheter 302 of a balloon catheter 300. Electrodes may be a variety of shapes and sizes. For example, an electrode may be square, rectangle, circle, oval, polygon, etc. Additionally, the electrodes may be positioned or spaced about the inflatable balloon 304 in a variety of ways. In the example shown in FIG. 3A, electrodes are spaced along the inflatable balloon 304 in a spiral pattern or helical pattern. In FIG. 3B, the electrodes are spaced laterally (i.e., lengthwise) along a length of the inflatable balloon 304. In FIG. 3C, the electrodes are spaced radially about a center C along a diameter D2 of the inflatable balloon 304. In FIG. 3D, the electrodes are spaced in a checkerboard pattern. The electrodes may be spaced symmetrically or asymmetrically relative to each other and/or features of the inflatable balloon. In an example, the electrodes may be spaced radially and lengthwise about the inflatable balloon 304 such that the balloon catheter need not be oriented or rotated to position electrodes relative to a phrenic nerve. Although specific configurations of electrodes are shown in FIGS. 3A-3D, other configurations are appreciated.

FIGS. 4A-4B show an anatomy of a patient 400 with placement of a balloon catheter 200 described herein. The elements of the patient 400 may be similar to that described for the patient 100 in FIG. 1. As shown, the body 402 of the patient 400 includes a right phrenic nerve 404A, a left phrenic nerve 404B, a diaphragm 406, and an esophagus 422. Although not shown, the body 402 of the patient 400 contains other anatomical structures, including a stomach fed by the esophagus 422.

Unlike FIG. 1, FIGS. 4A-4B show a balloon catheter 200 placed in an esophagus 422 in the body 402 of a patient 400. Features of the balloon catheter 200 are further described above in FIGS. 2A-2D. The balloon catheter 200 includes electrodes (not shown) capable of stimulating one or more phrenic nerves 404A, 404B. Additionally, the balloon catheter 200 includes a catheter and an inflatable balloon.

The balloon catheter 200 may be placed and secured in the body 402 by inflating the inflatable balloon. In the positions of the balloon catheter 200 shown in FIGS. 4A-4C, the inflatable balloon of the balloon catheter 200 is placed proximate a phrenic nerve (e.g., right phrenic nerve 404A or left phrenic nerve 404B, or both, as shown by dotted lines in FIGS. 4A-B). In the example shown in FIG. 4A, at least a portion of the inflatable balloon of the balloon catheter 200 is placed in the upper thoracic esophagus or in the mid-thoracic esophagus within a stimulation radius (e.g., stimulation radius R in FIG. 4C) of both the right phrenic nerve 404A and the left phrenic nerve 404B. In an alternative position shown in FIG. 4B, at least a portion of the inflatable balloon of the balloon catheter 200 is placed in the lower thoracic esophagus or abdominal esophagus within a stimulation radius (e.g., stimulation radius R in FIG. 4C) of both the right phrenic nerve 404A and the left phrenic nerve 404B and/or extensions of the right phrenic nerve 404A and/or the left phrenic nerve 404B along the diaphragm 406. In FIGS. 4A-4C, the balloon catheter 200 is in an inflated configuration. In the inflated configuration, the inflatable balloon of the balloon catheter 200 is inflated such as to secure or affix the inflatable balloon in the esophagus 422, as described herein.

The balloon catheter 200 is capable of providing voltage to one or more electrodes coupled to the inflatable balloon of the balloon catheter 200 from a power source. As described herein, attributes of each electrode along the balloon catheter 200 may be individually addressable and controllable by a controller (such as a PCB). In an example, a clinician may control nerve pacing of a nerve (e.g., right phrenic nerve 404A and/or left phrenic nerve 404B) via balloon catheter 200 from a controller, and observe the resultant ventilatory efforts of the patient at the ventilator. The controller may be a component of a ventilator, or may be a separate device. In an example where the controller is integrated into the ventilator, the power source is provided by the ventilator. In an example where the controller is a separate device, the controller may be associated with a display and user interface to allow viewing or selecting of electrode attributes.

To extend a magnetic field produced by the electrodes of the balloon catheter 200, an external pad may be placed on or near the skin of the patient, external to the patient. For example, the external pad may include one or more electrodes. The external pad may be moved relative to the body of the patient and/or relative to the balloon catheter 200 to provide more or less stimulation of a phrenic nerve of a patient.

FIG. 4C shows an example cross-sectional view of a balloon catheter 200 inflated inside an esophagus 422 of a patient 400, proximate a phrenic nerve 404A, at viewing plane P3. As shown, the inflatable balloon 208 of the balloon catheter 200 is inflated (otherwise referred to as in an inflated configuration). In the inflated configuration, the electrodes 210A-210C, 210Q are distributed inside the esophagus 422 along an interior wall of the esophagus 422. Additionally, in the inflated configuration, the inflatable annulus 218 is expanded to position at least one electrode 210A-B closer the phrenic nerve 404A and further from the catheter 206.

In the example shown in FIG. 4C, the phrenic nerve 404A may be effectively stimulated by a magnetic field resulting from a current running between a first electrode 210A and a second electrode 210B, with the current traveling inside a stimulation radius R of the phrenic nerve 404A. The current between two or more electrodes 210A-C, 210Q may be increased or decreased as required or desired. An increase in current may increase the amount of stimulation of the phrenic nerve 404A and/or increase the stimulation radius R of the phrenic nerve. A decrease in current may decrease the amount of stimulation of the phrenic nerve 404A and/or decrease the stimulation radius R of the phrenic nerve. The amount of stimulation of the phrenic nerve 404A may be changed based on patient efforts and/or desired or determined tidal volume. A stimulation pulse along two or more electrodes 210A-C, 210Q may be delivered over a pulse period to thereby cause stimulation of the phrenic nerve 404A during a pulse period. In an example, delivery of the stimulation pulse may be coordinated with delivery of an inhalation phase of ventilation provided by a mechanical ventilator (sometimes referred to as inhalation pacing).

In an example, electrodes 210A-L, 210Q that are positioned adjacent to each other along a radius and/or length of the balloon catheter 200 may be assigned opposite polarities (positive and negative, anode and cathode). Alternatively, two or more adjacent electrodes 210A-L, 210Q may be assigned the same polarity. In an instance, at least one remaining electrode 210A-L, 210Q is assigned the opposite polarity. The electrodes 210A-L, 210Q may be assigned charges and polarities to be interphasic (with charge accumulation inside the esophagus 422) or biphasic (little to no charge accumulation inside the esophagus 422).

As described herein, a phrenic nerve 404A is stimulated by a magnetic field resulting from an electrical current between two or more electrodes (e.g., electrodes 210A-B). If an electrode is a distance less than or equal to the stimulation radius R (which may be based on the strength of the resulting magnet field) from the phrenic nerve 404A, then the phrenic nerve 404A may be effectively stimulated by a current traveling to that electrode. In the example shown in FIG. 4C, two electrodes 210A-B are within the stimulation radius R of the phrenic nerve 404A. Thus, the electrodes 210A-B are positioned to effectively stimulate the phrenic nerve 404A. For example, a stimulation pulse between electrode 210A and either 210B, 210C, or 210Q may stimulate the phrenic nerve 404A. Additionally, a stimulation pulse between electrode 210B and either 210A, 210C, or 210Q may stimulate the phrenic nerve 404A. In the example shown, the most effective subset of electrodes to stimulate the phrenic nerve 404A is a stimulation pulse between electrodes 210A and 210B (the two electrodes positioned closest to the phrenic nerve) because the resulting magnetic field is generated closer to the phrenic nerve 404A. In an example, electrodes 210A and 210B may have opposite polarities while the remaining electrodes 210C and 210Q are not carrying a charge. In another example, all electrodes 210A-C, 210Q are assigned a charge.

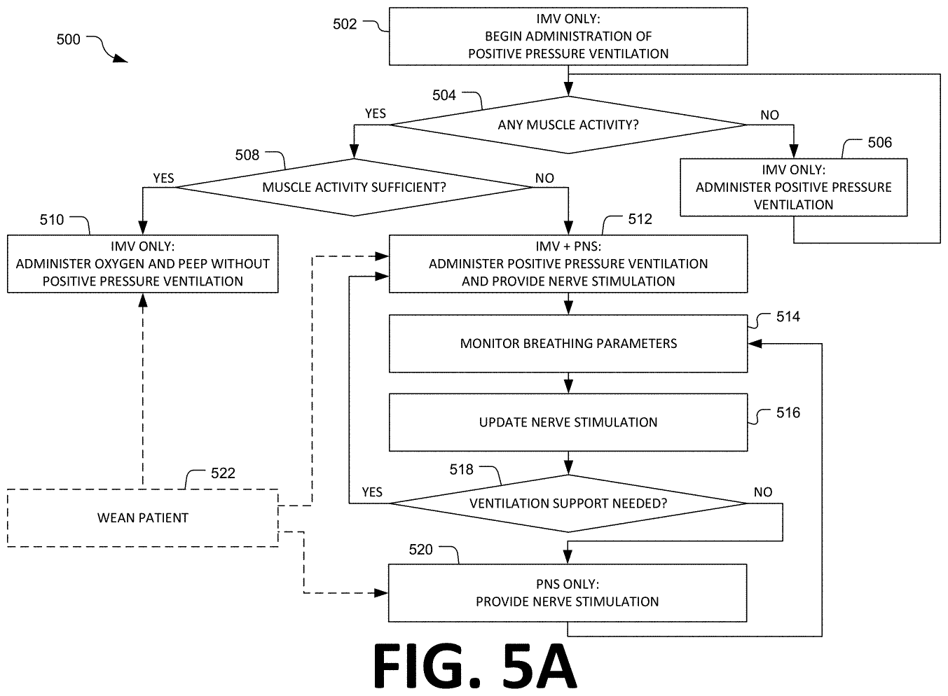

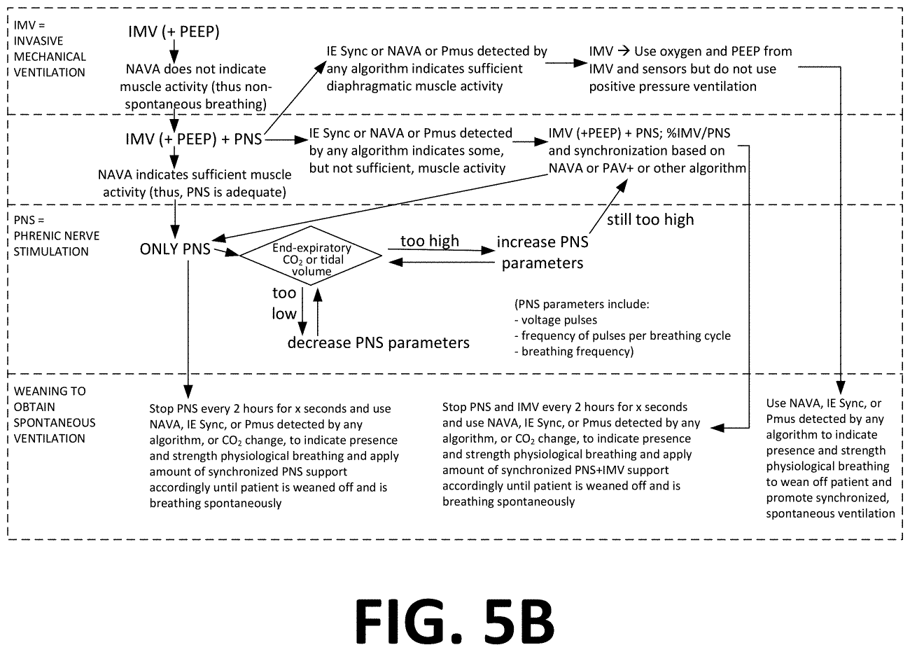

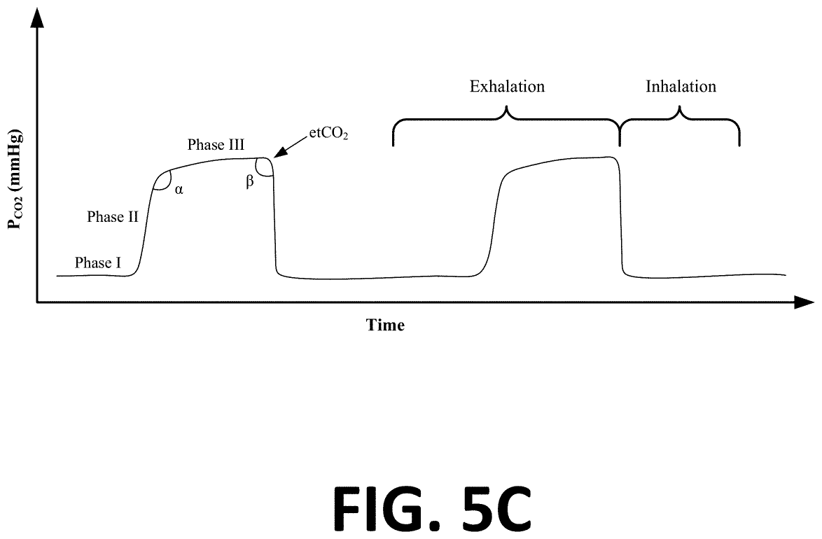

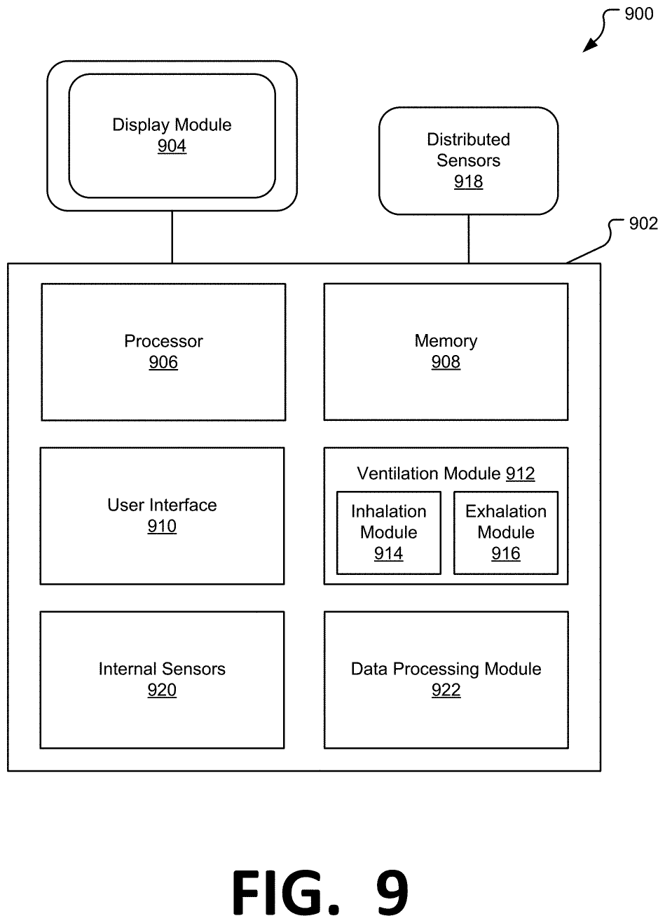

FIGS. 5-7 show example methods according to the technology described herein. The example methods include operations that may be implemented or performed by the systems and devices disclosed herein. For example, a ventilator as described either in FIG. 8 or FIG. 9 may perform the operations described in the methods. In addition, instructions for performing the operations of the methods disclosed herein may be stored in a memory of a ventilator (e.g., system memory 812 and 908 described in FIGS. 8-9). Further, reference to a balloon catheter in the example methods may be similar to aspects of the balloon catheter described herein (e.g., balloon catheter 200).

FIG. 5A shows an example method 500 for phrenic nerve stimulation. At operation 502, administration of positive pressure ventilation begins. At operation 502, positive pressure ventilation is administered via invasive mechanical ventilation (IMV) without phrenic nerve stimulation (otherwise referred to herein as IMV-only). During operation 502, a patient may exhibit little to no muscle activity and is not spontaneously breathing. Stated another way, the patient is not making an effort to breathe.

At determination 504, muscle activity is evaluated. If muscle activity of the patient (e.g., a patient effort) is not detected, the method flows to operation 506 where positive pressure ventilation without phrenic nerve stimulation (IMV-only) is administered. Operations 504-506 may repeat to periodically or continually monitor if any muscle activity is detected.

If, alternatively, some muscle activity is detected at determination 504, the method 500 flows to determination 508 where the muscle activity is evaluated. If the muscle activity is sufficient, then flow proceeds to operation 510 where oxygen and positive expiratory end pressure (PEEP) are administered without positive pressure ventilation and without phrenic nerve stimulation. The muscle activity is considered to be sufficient when the patient is effectively breathing on their own. The patient may then be weaned 522 from the ventilator.

If the muscle activity is detected, but is not sufficient, the method 500 flows to operation 512 where positive pressure ventilation is administered while providing phrenic nerve stimulation (PNS). The amount and frequency of PNS may be used to encourage patient muscle activity (otherwise referred to as assisted muscle activity or artificial muscle activity). By stimulating one or more phrenic nerves of the patient, the tidal volume and exhaled CO₂ of the patient is impacted. IMV may then be adjusted by the ventilator based on the measured muscle activity (otherwise referred to herein as the perceived patient effort), which includes the actual patient muscle activity (the patient's effort unassisted by PNS) combined with the artificial muscle activity. The patient may be weaned 522 from IMV and/or PNS at operation 512 by reducing an amount or frequency of positive pressure ventilation being administered over time and/or reducing an amplitude or frequency of phrenic nerve stimulation. A reduction in an amount or frequency of positive pressure ventilation being administered over time may be gradual. Additionally, a decrease in an amount or frequency of positive pressure ventilation may be supplemented with a change in amplitude or frequency of phrenic nerve stimulation (e.g., weaning from positive pressure ventilation without weaning from phrenic nerve stimulation). For example, an amplitude or frequency of phrenic nerve stimulation may be maintained or increased while reducing an amount or frequency of positive pressure ventilation. Alternatively, an amount or frequency of positive pressure ventilation and an amplitude or frequency of phrenic nerve stimulation may be reduced simultaneously (e.g., simultaneous weaning from positive pressure ventilation and phrenic nerve stimulation).

The amplitude and/or frequency of initial nerve stimulation may be based on training data. Alternatively, the amplitude and/or frequency of initial nerve stimulation may be based on a test, such as the test described below at operation 706 in method 700 in FIG. 7. Additionally, the amplitude and/or frequency of nerve stimulation may be started at low initial values and slowly increased over time and may be monitored with a feedback loop. The feedback loop may monitor tidal volume and/or end-tidal CO₂ (as further described below). The feedback loop may also monitor side effects of the patient caused by nerve stimulation, such as hiccups or smoothness of breathing. Each patient may respond to nerve stimulation differently.

In an example, PNS may be associated with the patient's efforts (e.g., coordinated in timing of delivery of stimulating pulse and/or amount of stimulation). In another example, the stimulating pulse may be timed according to a triggering strategy implemented by the ventilator during IMV. The triggering strategy may be associated with or based on the patient's efforts. The ventilator that is administering IMV may also provide PNS.

The stimulation may be coordinated with an inhalation phase of a patient. For example, a stimulating pulse may be coordinated with delivery of the flow and pressure to the patient during IMV. When delivering stimulating pulses according to a triggering strategy, the stimulating pulse may be delivered at the time of the trigger. In another example, the stimulation may be delayed from the trigger or may be delivered prior to the trigger. In another example, the stimulation may be delivered over a period of time that may include the time of the trigger. Delivery of the stimulation based on triggering allows pacing of diaphragm muscles in association with the trigger.

In a flow triggering strategy, the patient's inspiration effort is detected when the measured patient exhalation flow value drops below a flow baseline (i.e., the base flow) by a set amount (based on the triggering sensitivity). In a pressure triggering strategy or pressure trigger type, the patient's inspiration effort is detected when the measured expiratory pressure value drops below a pressure baseline (for example, the set PEEP level) by a set amount (based on triggering sensitivity). Another parameter that can be used for a triggering strategy trigger type is a derived signal, such as an estimate of the intrapleural pressure of the patient and/or the derivative of the estimate of the patient's intrapleural pressure. The term “intrapleural pressure,” as used herein, refers generally to the pressure exerted by the patient's diaphragm on the cavity in the thorax that contains the lungs, or the pleural cavity. The derivative of the intrapleural pressure value will be referred to herein as a “Psync” value that has units of pressure per time. An example of triggering and cycling based on the Psync value is provided in U.S. patent application Ser. No. 16/411,916 (“the '916 application”), titled “Systems and Methods for Respiratory Effort Detection Utilizing Signal Distortion” and filed on May 14, 2019, which is incorporated herein by reference in its entirety. That triggering strategy discussed in the '916 application is referred to herein as the “signal distortion” triggering strategy or “signal distortion” trigger type. As discussed in the '916 application, the signal distortion triggering strategy may operate on the Psync signal or other signals, such as flow or pressure.

At operation 514, breathing parameters of the patient are monitored. Breathing parameters may include tidal volume and/or end-tidal CO₂. These breathing parameters may be determined or measured by the ventilator administering positive pressure ventilation to the patient. The breathing parameters may be used to indicate or evaluate muscle activity of the patient.

At operation 516, nerve stimulation is updated. The nerve stimulation may be updated based on the breathing parameters monitored at operation 514. Depending on the breathing parameters, an aspect of the nerve stimulation may be adjusted. For example, if end-tidal CO₂ is too high, stimulation may be increased. The stimulation may be increased by increasing an amplitude of stimulation and/or by increasing a frequency of stimulation. In another example, if the end-tidal CO₂ is too low, stimulation may be decreased. The stimulation may be decreased by decreasing an amplitude of stimulation and/or by decreasing a frequency of stimulation.

As another example, nerve stimulation may be adjusted based on the following table:

TABLE 1

| Condition | Qualifier | Update to Nerve Stimulation |
|---|---|---|
| CO₂ signal loss, | Increase in alpha | No changes in rate or amplitude |
| presence of poor | angle greater than | command |
| signal, or change | 100 degrees | |
| in V/Q | | |
| etCO₂ = 35-45 | Presence of all | No changes in rate or amplitude |
| mmHg | phases of | command |
| | capnogram and | |
| | alpha angle less | |
| | than 100 degrees | |
| etCO₂ > 45 mmHg | Presence of | Increase rate by 2/min, wait for |
| (Elevated etCO₂) | Phase III | 5 minutes before next change |
| etCO₂ < 35 mmHg | Presence of | Decrease rate by 2/min, wait for |
| (Reduced etCO₂) | Phase III | 5 minutes before next change |
| etCO₂ = 25-55 | Absence of | Increase amplitude by 10%, wait |
| mmHg | Phase III | for 3 minutes before next change |
| | | (if no change, increase rate by |
| | | 2/min) |

Aspects of Table 1 are further described with respect to FIG. 5C. FIG. 5C shows an example capnography. The example capnography is a graph of partial pressure of CO₂ exhaled versus time. Exhalation includes three phases: Phase I, Phase II, and Phase III. Phase I is the first portion of exhalation involving dead space. The partial pressure of CO₂ during Phase I should be negligible because no CO₂ has been exhaled with exhalation of the dead space. Phase II is the initial mixing of dead space (which does not include CO₂) and alveolar gas (which contains CO₂). The partial pressure of CO₂ during Phase II is a positive trend. Phase III is a plateau during exhalation which consists mostly of alveolar gases. The last point in time of Phase III defines the end-tidal CO₂ (etCO₂). The angle between Phase II and Phase III is the alpha (a) angle, which represents a patient's alveolar-emptying efficiency. A patient within normal physiological parameters traditionally has an alpha angle of approximately 100 degrees. The angle between Phase III and the beginning of inhalation is the beta (β) angle. The beta angle may be used to assess rebreathing of the patient. Rebreathing is often associated with a beta angle greater than 90 degrees.

Turning to the example nerve stimulation updates shown in Table 1, a condition and a qualifier may be evaluated to determine an adjustment to the nerve stimulation. The nerve stimulation may be adjusted to achieve a desired oxygen absorption by the patient (for which breathing is sufficient to safely sustain the patient). For example, if a CO₂ signal loss is detected, a poor signal is present, or a change is detected in ventilation-perfusion ratio (V/Q) and the alpha angle is greater than 100 degrees, then the nerve stimulation may be maintained without being updated. If the end-tidal CO₂ is between 35-45 mmHg with an alpha angle less than 100 degrees and with a presence of Phase I, Phase II, and Phase III, then nerve stimulation may be maintained without being updated. If end-tidal CO₂ is elevated (e.g., above 45 mmHg) and Phase III is present, the nerve stimulation may be updated to increase the frequency of stimulation. For instance, the rate of nerve stimulation may be increased by two stimulating pulses per minute. If end-tidal CO₂ is reduced (e.g., below 35 mmHg) and Phase III is present, the nerve stimulation may be updated to reduce the frequency of stimulation. For instances, the rate of nerve stimulation may be decreased by two stimulating pulses per minute. In an example where the frequency or rate of nerve stimulation changes, a wait period may be enacted for a subsequent change to the nerve stimulation. For example, if a rate of nerve stimulation changes, the wait period before the nerve stimulation may be change again may be five minutes. If the end-tidal CO₂ is between 25-55 mmHg and there is an absence of Phase III, then the amplitude of the nerve stimulation may be updated. In an instance, the amplitude of the nerve stimulation is increased. The amplitude of the nerve stimulation may be increased by 10%. A wait period may be enacted based on a change in amplitude. For example, a wait period may be three minutes. In an example, if the end-tidal CO₂ is between 25-55 mmHg, there is an absence of Phase III, and an increase in amplitude after a wait period causes no change, then the rate of nerve stimulation may be increased.

The amplitude and or frequency of nerve stimulation may have a maximum. For example, maximums on amplitude and/or frequency of nerve stimulation may be set by a ventilator and/or a clinician for the safety of the patient and/or for patient comfort.

At determination 518, it is determined if ventilation support is required or desired. This determination may be made by the ventilator administering positive pressure ventilation. In an example, the ventilator may provide ventilation support to the patient according to a support mode. The support mode may be a patient-directed mode that adjusts ventilation based on a change in the patient's demand and/or a patient's work of breathing (WOB), such as neurally adjusted ventilatory assist (NAVA™) or proportional assist ventilation (PAV™/PAV™+). In this way, the ventilator provides complementary support to the patient to account for the proportional amount of effort not completed by the patient (to complete a sufficient breath). An example of methods and systems for providing proportional assist ventilation are discussed in U.S. Pat. No. 10,806,879, filed May 14, 2018, titled Methods and Systems for an Optimized Proportional Assist Ventilation, which is hereby incorporated by reference in its entirety.

When providing ventilation support, the ventilator is unaware of artificial muscle activity that is caused by nerve stimulation. Thus, ventilation support at determination 518 is evaluated based on the breathing parameters of the patient as influenced by any nerve stimulation (otherwise referred to herein as perceived patient effort, as perceived by the ventilator).

If ventilation support is needed, the method 500 may repeat operations 512-518. The positive pressure ventilation may be administered based on the ventilation support that is needed, associated with determination 518, while continuing to provide nerve stimulation. The breathing parameters of the patient are monitored, nerve stimulation is updated, and ventilation support is re-evaluated.

If, alternatively, ventilation support is not needed, the method flows to operation 520 where nerve stimulation is provided without administering positive pressure ventilation (PNS-only). The nerve stimulation at operation 520 may be based on a breathing parameter of the patient, as monitored periodically. For example, operations 514-520 may repeat until the patient is weaned 522 from positive pressure ventilation and nerve stimulation (e.g., until the patient is breathing on their own, unassisted). Thus, the patient may be weaned 522 from nerve stimulation over time.

FIG. 5B shows an example flowchart for phrenic nerve stimulation in a different format than FIG. 5A. In the flowchart shown in FIG. 5B, a patient begins on IMV and stays on IMV until the ventilator determines that muscle activity is present for the patient. After muscle activity is detected, nerve stimulation is added to support the patient in addition to IMV. The ventilator may provide ventilatory support to the patient as required or desired (in addition to the ventilator support caused by nerve stimulation), based on the perceived patient effort. If no ventilatory support is required or desired in addition to the nerve stimulation, then nerve stimulation is provided without IMV. Breathing parameters of the patient are monitored, such as end expiratory CO₂ and/or tidal volume. Nerve stimulation parameters are adjusted based on the breathing parameters. Nerve stimulation parameters may include amplitude (e.g., amount of voltage provided to one or more electrodes) and frequency (e.g., number of stimulation pulses per time, such as per breathing cycle or per minute). The patient may be weaned from nerve stimulation based on a hold maneuver. For example, nerve stimulation may be paused or skipped for a hold period (e.g., one to three breath cycles or five to thirty seconds) to measure a presence and/or strength of unassisted patient breathing. The nerve stimulation may then be adjusted according to the unassisted/actual patient effort. The adjustment of patient effort may be performed over time to ultimately result in the patient being weaned from nerve stimulation.

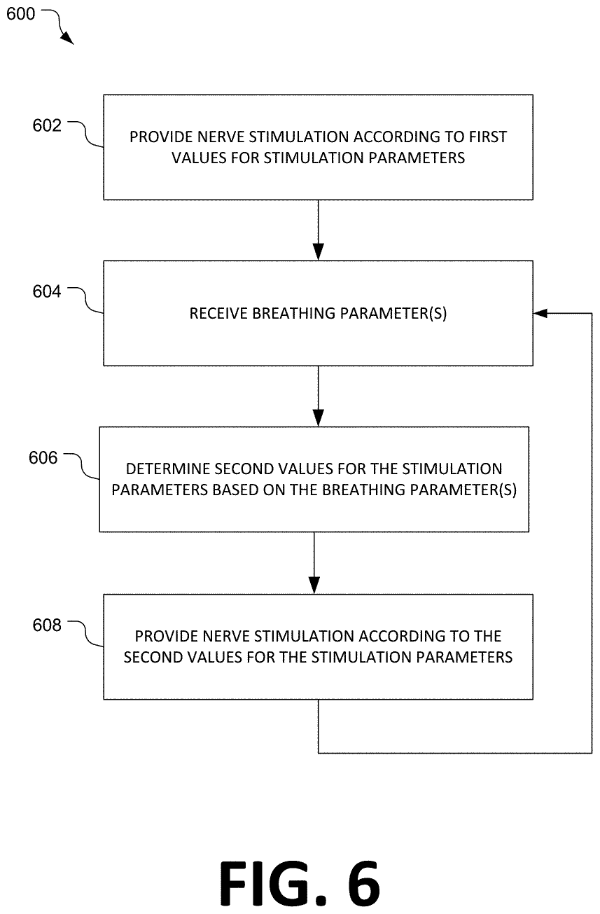

FIG. 6 shows another example method for phrenic nerve stimulation. At operation 602, nerve stimulation is provided according to first values for stimulation parameters. As described herein, delivery of a stimulating pulse to electrodes near a nerve stimulates the nearby nerve. Delivery of the stimulating pulse may be caused by providing a voltage to the electrodes on a balloon catheter secured inside a vessel in a patient. The voltage provided to two or more electrodes results in a current between the two electrodes. The current and resulting magnetic field result in stimulation of the nearby nerve.

As further described above, the stimulation parameters may include an amplitude (i.e., amount of voltage delivered to two or more electrodes and/or amount of current between two or more electrodes) and a frequency (i.e., frequency or rate of stimulation pulses over a time period). The first values for the stimulation parameters may be universal for any patient or set at initial values based on patient's body weight or other physical factors. Alternatively, the first values for the stimulation parameters may be based on a test, such as the test described below at operation 706 in method 700 in FIG. 7. As another alternative, the first value for the nerve stimulation may be based on a patient parameter. The patient parameter may be identified by the ventilator (e.g., measured, calculated, determined, etc.) or may be provided by a clinician. The patient parameter may be a breathing mode (e.g., spontaneous breath mode or mandatory breath mode), respiratory rate, predicted body weight, patient effort, tidal volume, inspiratory time, expiratory time, peak inspiratory flow, peak circuit pressure, flow pattern, PEEP, lung compliance, lung resistance, or any other patient parameter associated with a breathing pattern of a patient and/or a patient's diaphragm muscle activity not limited to the aforementioned list. Additionally or alternatively, the first value of the phrenic nerve stimulation may be based on a location of stimulation (e.g., which nerve(s) are stimulated by the nerve stimulation and where in the body), an amount or location of electrodes providing the nerve stimulation (e.g., which electrodes are provided voltage), an attribute of an electrode (as further described herein), etc.

Additionally, the nerve stimulation parameter may be associated with the current ventilation settings and/or current breath delivery performed by the ventilator. The nerve stimulation parameter may change with a change in the patient parameter. For example, the timing of spontaneous breaths and/or mandatory breaths may be coordinated with delivery of stimulation pulses, thus the stimulation rate may change in association with breathing mode. Additionally or alternatively, the amount of stimulation may vary based on the breathing mode. For example, the amount of stimulation may be specified in association with mandatory breaths. In another example, the amount of stimulation may vary from breath-to-breath in association with spontaneous breaths based on the patient's effort and/or length and/or frequency of inhalation.

In another example, updated ventilation settings may be determined based on the patient parameter and the nerve stimulation parameter. In an example, the ventilator may determine the updated ventilation settings. In another example, the updated ventilation settings may be associated with an increase or a decrease in at least one of: the inhalation flow or the inhalation pressure. With an increase in perceived patient effort, the inhalation flow and/or the inhalation pressure may be decreased. The updated ventilation settings may be based on a patient parameter and adjusted based on the determined nerve stimulation parameter. In a further example, the settings difference between the initial ventilation settings and the updated ventilation settings is based on the nerve stimulation parameter.

At operation 604, a breathing parameter is received. Multiple breathing parameters may be received. In an example, the breathing parameter is end-tidal CO₂ and/or tidal volume. The breathing parameter may be measured by a ventilator coupled to the patient that was provided nerve stimulation at operation 602. The received breathing parameter may be agnostic to the nerve stimulation provided. For example, the breathing parameter may be influenced by stimulation of nerve.

At operation 606, second values for the stimulation parameters are determined, based on the breathing parameter received at operation 604. The stimulation parameters may be determined by the ventilator or may be determined by a clinician. As further described above, the amplitude and/or the frequency of stimulation may be adjusted or maintained based on breathing parameters of the patient. The stimulation parameters may be increased if the patient is not receiving enough oxygen (e.g., a tidal volume is too low and/or end-tidal CO₂ is too high) and the stimulation parameters may be decreased if the patient is receiving too much oxygen (e.g., tidal volume is too high and/or end-tidal CO₂ is too low). At operation 608, nerve stimulation is provided according to the second values for the stimulation parameters.

As required or desired, the method 600 may repeat operations 604-608 as breathing parameters of the patient are received or monitored over time. For example, a change in the breathing parameter may result in a different value (e.g., third value) for the stimulation parameters which are then provided, which may then cause a change in the breathing parameter.

FIG. 7 shows another example method for phrenic nerve stimulation. In an example, the method 700 of FIG. 7 may be applied to determine proper placement of a balloon catheter and/or to optimize one or more attributes of each electrode of the balloon catheter.

At operation 702, a balloon catheter may be positioned in a vessel. During positioning, an inflatable balloon of the balloon catheter may be in a deflated configuration. The balloon catheter includes a set of electrodes coupled to the inflatable balloon. The set of electrodes may be spaced lengthwise or radially about the inflatable balloon. Each electrode may have similar characteristics of electrodes described herein. In an example, the vessel may be an esophagus of a patient, least a portion of which is located adjacent a phrenic nerve.

At operation 704, the inflatable portion is inflated to secure the balloon catheter in the vessel. As further described herein, inflating the inflatable portion (e.g., inflatable balloon 208) may secure the balloon catheter in the vessel by expanding a diameter of the inflatable portion until an exterior surface of the inflatable portion contacts an interior wall of the vessel. The pressure and frictional forces between the inflatable portion and the interior wall of the vessel may secure the balloon catheter related to the vessel.

At operation 706, delivery of a test stimulating pulse to at least two test electrodes in the set of electrodes may be caused. The delivery of the test stimulating pulse may be caused by a controller. The controller may be commanded by a ventilator or a clinician. Aspects of the stimulating pulse (e.g., stimulation frequency, amount of stimulation, etc.) or attributes of the at least two test electrodes may be controlled independently, as further described herein. The ventilator may perform a negative inspiratory force (NIF) maneuver while delivering the stimulating pulse. The NIF maneuver is a coached maneuver where the patient is prompted to draw a maximum inspiration against an occluded airway (when the inhalation valve and exhalation valve are fully closed). Performing the NIF maneuver while delivered the stimulating pulse may allow more accurate estimations of the force generated by the stimulating pulse. Better estimations of the force generated by the stimulating pulse may assist in determination of the balloon catheter's placement in the body, selection of electrodes used to stimulate the phrenic nerve, or any other pacing parameter.

The initialization technique may test a variety of attributes of each electrode in variety of combinations. The optimization of attributes may be based on the amount of stimulation of the phrenic nerve or the tidal volume of the patient during phrenic nerve stimulation. In an example, a subset of the electrodes 210A-210L, 210Q may not be assigned an attribute (e.g., may not be used to stimulate the phrenic nerve). The initialization technique may be performed manually or using a predetermined program while monitoring the effort on the patient to determine the optimum settings.

In an example, an initialization technique may be carried out as follows. During or after placement of the balloon catheter in the body, one or more different subsets of electrodes are separately pulsed and the effect is observed and stored. After the subsets of electrodes have been pulsed, their effects on stimulation of the phrenic nerve are compared. In an example, the comparison may include an amount of diaphragm muscle movement and/or relative tidal volumes caused by the phrenic nerve stimulation. A subset of electrodes with a large effect (e.g., large amount of diaphragm muscle movement and/or large tidal volume) are identified. The identified subset of electrodes may be used to stimulate the phrenic nerve. In an example, a subset of electrodes may be determined to effectively stimulate the phrenic nerve when delivering a stimulation pulse.

The controller may implement the delivery of test stimulating pulses via an automatic algorithm. The automatic algorithm may sequentially provide power to various electrode combinations, as described herein. Each combination may be assessed to determine which combination has highest efficacy (e.g., effective nerve stimulation) without side effects (e.g., hiccups). The automatic algorithm may also test increased and/or decreased stimulation amounts to assess efficacy related to the attributes of a combination of electrodes. For example, the automatic algorithm may test a set of combinations of electrodes at a first amplitude of stimulation and then test the same set of combinations of electrodes at a second amplitude of stimulation. Alternatively, the automatic algorithm may test a set of combinations of electrodes at a first amplitude to determine a desired combination of electrodes and then test the desired combination of electrodes at different amplitudes of stimulation (e.g., slowly increase the amplitude of stimulation until effective stimulation is achieved to minimize side effects).

At determination 708, effective stimulation of the phrenic nerve is evaluated. For example, a clinician or surgeon may determine if the phrenic nerve was effectively stimulated during delivery of the test at operation 706. In an example, a patient's bodily reaction to the stimulating pulse may be used in the determination. For example, a clinician may evaluate feedback of the patient's diaphragm muscles in response to the stimulating pulse. Alternatively, a ventilator may determine if the phrenic nerve was effectively stimulated during the test stimulating pulse. For example, the ventilator may receive data associated with the patient's effort or tidal volume caused by the test stimulating pulse. The evaluation of effective stimulation, by a human or the ventilator, may be obtained using visual information (e.g., rise and fall of the patient's chest), a determined pressure and/or flow in the breathing circuit, diaphragm muscle activity information, or any other information related to stimulation of the phrenic nerve to cause stimulation of diaphragm muscles.

In examples, the determination 708 is binary (e.g., whether the phrenic nerve has been effectively stimulated) or may be based on a threshold (e.g., whether the stimulating pulse caused at least a specified amount of stimulation of the phrenic nerve). If it is determined that the phrenic nerve is not effectively stimulated, flow branches to “NO” and operations 702-708 may be repeated. For example, the balloon catheter may be repositioned in the vessel until the test stimulating pulse effectively stimulates the nerve. In examples, this process may be referred to as mapping an effective position of the balloon catheter in the vessel. In examples, the test stimulating pulse may be continuous while operations 702-708 repeat.

If, however, it is determined that the phrenic nerve is effectively stimulated, flow branches “YES” to operation 710. At operation 710, nerve stimulation is provided according to the stimulation parameters. Aspects of providing nerve stimulation may be similar to, or the same as, one or more operations described in method 600 in FIG. 6 or otherwise described herein. For example, aspects of the stimulating pulse (e.g., timing, amount, etc.) and/or attributes of the set of electrodes may be controlled by a ventilator. In an example, a ventilator may coordinate delivery of stimulating pulses with patient parameters and/or ventilation settings. In a further example, delivery of the stimulating pulse may be associated with an inhalation phase of a patient being actively ventilated by a ventilator. Alternatively, aspects of the stimulating pulse (e.g., timing, amount, etc.) may be controlled by another device and/or a clinician.

One or more operations of the method 700 may be repeated as required or desired. For example, operations 706-710 may be repeated after a change to a patient's environment. For example, operations 706-710 may repeat if a patient's medication (e.g., anesthesia) is changed or modified.

FIG. 8 is a diagram illustrating an example of a ventilator 800 connected to a patient 850 and a balloon catheter. Ventilator 800 includes a pneumatic system 802 (also referred to as a pressure generating system 802) for circulating breathing gases to and from patient 850 via the ventilation tubing system 830, which couples the patient to the pneumatic system via an invasive (e.g., endotracheal tube, as shown) or a non-invasive (e.g., nasal mask) patient interface.

Ventilation tubing system 830 may be a two-limb (shown) or a one-limb circuit for carrying gases to and from the patient 850. In a two-limb example, a fitting, typically referred to as a “wye-fitting” 870, may be provided to couple a patient interface 880 to an inhalation limb 834 and an exhalation limb 832 of the ventilation tubing system 830.

Pneumatic system 802 may have a variety of configurations. In the present example, pneumatic system 802 includes an exhalation module 808 coupled with the exhalation limb 832 and an inhalation module 804 coupled with the inhalation limb 834. Compressor 806 or other source(s) of pressurized gases (e.g., air, oxygen, and/or helium) is coupled with inhalation module 804 to provide a gas source for ventilatory support via inhalation limb 834. Stimulation control module 818 may provide voltage to a balloon catheter (e.g., to independently control electrodes on the balloon catheter) as described herein. The pneumatic system 802 may include a variety of other components, including mixing modules, valves, sensors, tubing, accumulators, filters, etc.

Controller 810 is operatively coupled with pneumatic system 802, signal measurement and acquisition systems, and an operator interface 820 that may enable an operator to interact with the ventilator 800 (e.g., change ventilator settings, select operational modes, view monitored parameters, etc.). Controller 810 may include memory 812, one or more processors 816, storage 814, and/or other components of the type found in command and control computing devices. In the depicted example, operator interface 820 includes a display 822 that may be touch-sensitive and/or voice-activated, enabling the display 822 to serve both as an input and output device.

The memory 812 includes non-transitory, computer-readable storage media that stores software that is executed by the processor 816 and which controls the operation of the ventilator 800. In an example, the memory 812 includes one or more solid-state storage devices such as flash memory chips. The processor 816 may be configured to control attributes of the electrodes on a balloon catheter. In an alternative example, the memory 812 may be mass storage connected to the processor 816 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 816. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilatory system or between the ventilatory system and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

FIG. 9 is a block-diagram illustrating an example of a ventilatory system 900. Ventilatory system 900 includes ventilator 902 with its various modules and components. That is, ventilator 902 may further include, among other things, memory 908, one or more processors 816, user interface 910, and ventilation module 912 (which may further include an inhalation module 914 and an exhalation module 916). Memory 908 is defined as described above for memory 908. Similarly, the one or more processors 906 are defined as described above for one or more processors 906. Processors 906 may further be configured with a clock whereby elapsed time may be monitored by the ventilatory system 900.

The ventilatory system 900 may also include a display module 904 communicatively coupled to ventilator 902. Display module 820 (otherwise referred to as operator interface 820) provides various input screens, for receiving input, and various display screens, for presenting useful information. Inputs may be received from a clinician. The display module 820 is configured to communicate with user interface 910 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., ventilatory data, alerts, patient information, parameter settings, modes, etc.). The elements may include controls, graphics, charts, tool bars, input fields, icons, etc. Alternatively, other suitable means of communication with the ventilator 902 may be provided, for instance by a wheel, keyboard, mouse, or other suitable interactive device. Thus, user interface 910 may accept commands and input through display module 904. Display module 904 may also provide useful information in the form of various ventilatory data regarding the physical condition of a patient and/or a prescribed respiratory treatment. The useful information may be derived by the ventilator 902, based on data collected by a data processing module 922, and the useful information may be displayed in the form of graphs, wave representations (e.g., a waveform), pie graphs, numbers, or other suitable forms of graphic display. For example, the data processing module 922 may be operative to determine ventilation settings (otherwise referred to as ventilatory settings, or ventilator settings) associated with a balloon catheter for nerve stimulation, etc., as detailed herein.

Ventilation module 912 may oversee ventilation of a patient according to ventilation settings. Ventilation settings may include any appropriate input for configuring the ventilator to deliver breathable gases to a particular patient, including measurements and settings associated with exhalation flow of the breathing circuit. Ventilation settings may be entered, e.g., by a clinician based on a prescribed treatment protocol for the particular patient, or automatically generated by the ventilator, e.g., based on attributes (i.e., age, diagnosis, ideal body weight, predicted body weight, gender, ethnicity, etc.) of the particular patient according to any appropriate standard protocol or otherwise, such as may be determined in association with stimulating a phrenic nerve with a balloon catheter. In some cases, certain ventilation settings may be adjusted based on the exhalation flow, e.g., to optimize the prescribed treatment.

Ventilation module 912 may further include an inhalation module 914 configured to deliver gases to the patient and an exhalation module 916 configured to receive exhalation gases from the patient, according to ventilation settings that may be based on the exhalation flow. As described herein, inhalation module 914 may correspond to the inhalation module 804 and 914, or may be otherwise coupled to source(s) of pressurized gases (e.g., air, oxygen, and/or helium), and may deliver gases to the patient. As further described herein, exhalation module 916 may correspond to the exhalation module 808 and 916, or may be otherwise coupled to gases existing the breathing circuit.

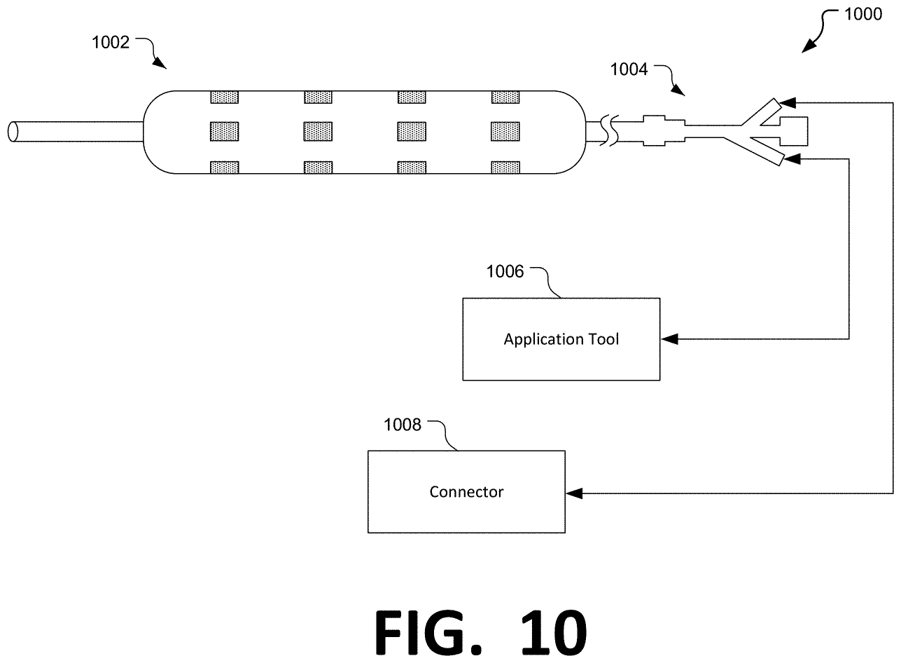

FIG. 10 is an example system 1000 including a balloon catheter 1002, an application tool 1006, and a connector 1008. The balloon catheter 1002 shares aspects with the balloon catheter described herein (e.g., balloon catheter 200). The application tool 1006 is any tool known in the art capable of assisting with inserting, implanting, removing, or otherwise moving and/or securing the balloon catheter 1002 within the body of the patient while the application tool 1006 remains outside of the body of the patient. The application tool 1006 enables inflation and deflation of an inflatable balloon of the balloon catheter (e.g., to transition between an inflated configuration and a deflated configuration via the balloon port of a connector 1004 of the balloon catheter 1002, as described herein).

The connector 1008 may be any connector known in the art capable of including independent leads (e.g., wires) for one or more of the electrodes on the balloon catheter 1002. The connector 1008 may couple to a power port of a connector 1004 of the balloon catheter 1002. Each lead of the connector 1008 may independently energize the electrode to which the lead is electrically coupled. For example, if the balloon catheter 1002 has eight electrodes, the connector 1008 may have eight independent leads electrically coupled to each of the eight electrodes. The connector 1008 may be electrically couplable to a controller to independently energize each electrode on the balloon catheter 1002 at each lead. The controller may be a component of a ventilator (e.g., stimulation control module 818 of ventilator 800). Alternatively, the controller may be a stand-alone nerve stimulation controller or may be incorporated into some other device such as a patient monitor. If an external pad is used, the same or different controller and/or connector 1008 may be used to control electrodes in the external pad.

Although the present disclosure discusses the implementation of these techniques in the context of phrenic nerve stimulation, stimulation of any nerve or nerves in the body of a patient is appreciated. Additionally, although the balloon catheter for nerve stimulation is described as being placed in an esophagus of a patient, placement of the balloon catheter in any orifice in the body is appreciated. For example, the balloon catheter can be adapted for any vein or tube in body to excite any nerve nearby. Further, the techniques introduced above may be implemented for a variety of medical devices or devices utilizing nerve stimulation. A person of skill in the art will understand that the technology described in the context of a medical ventilator for human patients could be adapted for use with other systems such as ventilators for non-human patients or general gas transport systems. Additionally, a person of ordinary skill in the art will understand that the modeled exhalation flow may be implemented in a variety of breathing circuit setups.

Those skilled in the art will recognize that the methods and systems of the present disclosure may be implemented in many manners and as such are not to be limited by the foregoing aspects and examples. In other words, functional elements being performed by a single component or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A balloon catheter comprising:
a catheter;
an inflatable balloon positioned along a length of the catheter, the inflatable balloon having a surface;
a set of electrodes on the surface of the inflatable balloon, the set of electrodes including a first electrode and a second electrode, the set of electrodes capable of stimulating a nerve; and
at least one wire capable of providing power to the set of electrodes.

2. The balloon catheter of claim 1, wherein the first electrode and the second electrode are spaced apart on the surface of the inflatable balloon.

3. The balloon catheter of claim 2, wherein the first electrode and the second electrode are spaced apart radially on the surface of the inflatable balloon.

4. The balloon catheter of claim 1, the balloon catheter further comprising:
a balloon port to inflate the inflatable balloon;
an injection port to introduce a substance into the catheter; and
a power port electrically coupled to the set of electrodes via the at least one wire.

5. The balloon catheter of claim 1, wherein the set of electrodes further includes a third electrode and a fourth electrode.

6. The balloon catheter of claim 5, wherein the balloon catheter further includes four of the set of electrodes.

7. The balloon catheter of claim 6, wherein the four sets of electrodes are distributed along the surface of the inflatable balloon.

8. The balloon catheter of claim 1, wherein the first electrode and the second electrode are independently controllable.

9. A method for stimulating a nerve, comprising:
providing first nerve stimulation to a patient under mechanical ventilation, wherein the first nerve stimulation is based on first values for a set of stimulation parameters;
after providing the first nerve stimulation to the patient, receiving a breathing parameter of the patient;
determining second values for the set of stimulation parameters, based on the breathing parameter; and
providing second nerve stimulation to the patient, based on the second values for the set of stimulation parameters.

10. The method of claim 9, wherein the set of stimulation parameters includes a frequency of stimulation, or an amplitude of stimulation, or both.

11. The method of claim 10, wherein the breathing parameter of the patient includes at least one of a tidal volume of the patient, an end-tidal CO₂, or a patient effort.

12. The method of claim 10, the method further comprising:
determining that the breathing parameter exceeds a threshold; and
based on determining that the breathing parameter exceeds the threshold, changing at least one of the frequency of stimulation or the amplitude of stimulation.

13. The method of claim 9, wherein the method further comprises:
implementing a weaning maneuver, wherein the weaning maneuver includes:
pausing providing nerve stimulation; and
during the pause, receiving an indication of a patient effort, wherein the second values for the set of stimulation parameters is based on the patient effort.

14. The method of claim 9, wherein the breathing parameter of the patient is based on the stimulation parameters, and wherein the first nerve stimulation and the second nerve stimulation stimulate a phrenic nerve and are provided from inside an esophagus of the patient.

15. The method of claim 9, the method further comprising:
based on the breathing parameter, reducing delivery of ventilation while maintaining phrenic nerve stimulation.

16. The method of claim 9, wherein a frequency of the phrenic nerve stimulation is based on the detected patient effort.

17. A ventilator for providing mechanical ventilation and nerve stimulation to a patient, the ventilator comprising:
a display;
an inhalation port for providing breathing gas to the patient;
a nerve stimulation port;
a processor;
memory storing instructions that, when executed by the processor, cause the ventilator to perform a set of operations comprising:
detecting a perceived patient effort;
delivering the breathing gas to the patient via the inhalation port, based on the perceived patient effort;
providing power to a set of electrodes coupled to an esophageal tube via the nerve stimulation port, wherein the power has a frequency and an amplitude;
measuring a breathing parameter of the patient, wherein the breathing parameter includes a tidal volume of the patient, an end-tidal CO₂ of the patient, or both; and
based on the breathing parameter of the patient, adjusting at least one of the frequency, the amplitude, or both.

18. The ventilator of claim 17, the set of operations further comprising:
pausing providing power to the set of electrodes; and
while pausing providing power, detecting an unassisted patient effort, wherein adjusting the at least one of the frequency, the amplitude, or both is further based on the unassisted patient effort.

19. The ventilator of claim 17, wherein adjusting at least one of the frequency, the amplitude, or both is further based on the perceived patient effort.

20. The ventilator of claim 17, wherein the esophageal tube includes an inflatable balloon and wherein the set of electrodes is positioned along the inflatable balloon.
